(19) United States
(12) Patent Application Publication
(10) Pub. No.: US 2006/0058283 A1
(43) Pub. Date: Mar. 16, 2006

(54) Compositions comprising ubiquinones

(75) Inventor: Bruce H. Lipshutz, Goleta, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Zymes, Inc., Goleta, CA (US)

(21) Appl. No.: 10/973,158

(22) Filed: Oct. 25, 2004

Related U.S. Application Data
(60) Provisional application No. 60/610,304, filed on Sep. 15, 2004.

(51) Int. Cl. A61K 31/397; A61K 31/12

(52) U.S. Cl. 514/210.02; 514/690

(57) ABSTRACT

The present invention provides antihyperlipidemic or antihypercholesterolemic compositions. These compositions ameliorate the adverse effects associated with other antihyperlipidemic or antihypercholesterolemic compositions. The present invention further provides methods of using the antihyperlipidemic or antihypercholesterolemic compositions.

### FIELD OF THE INVENTION

The present invention pertains to ubiquinone, e.g., Coenzyme Q₁₀, compositions and methods of using such compositions for the treatment and prevention of hypercholesterolemia.

### BACKGROUND OF THE INVENTION

Despite advances and new therapies, cardiovascular disease, principally heart disease and stroke, is the leading cause of death in the United States for both men and women across all racial and ethnic groups. As recently as 2001, it was estimated that 64 million Americans have one or more forms of cardiovascular disease. Nearly 50 million have high blood pressure, 13.2 million have coronary heart disease, including myocardial infarction and angina pectoris, and 4.8 million suffered a stroke. American Heart Association at http://www.americanheart.org.

Coronary heart disease is generally caused by atherosclerosis, the narrowing of the coronary arteries due to build up of plaque. Cholesterol lowering drugs, including HMG-CoA reductase inhibitors (“statins”), decreases atheromotous plaque buildup in subjects taking such medications. However, these drugs also have side effects including damaging skeletal muscle (e.g. myopathy, muscle soreness, rhabdomyolysis), deleterious endocrine effects (e.g. decline in adrenal or gonadal hormone production), and impairment of renal and liver function. In some subjects, side effects can be so severe as to necessitate discontinuation of the medication, placing the subject at increased risk of cardiovascular disease. Improved therapies are needed.

### SUMMARY OF THE INVENTION

Skeletal muscle weakness is a recognized adverse effect of statins. The statins are oftentimes associated with some degree of myalgia, myopathy (i.e. muscle pain, tenderness or weakness with elevated creatine phosphokinase (CPK) values) and rhabdomyolysis. See, for example. Ho et al., 2004, Am. J. Emerg. Med. 22(3):234-5; Jamil and Iqbal, 2004, Heart 90(1): e3; Daugirl and Crowell, 2003, J. Fam. Pract. 52(12):973-7; Luh and Karnath, 2003, JAMA, 289(13): 1681-90; Thompson et al., 2003, JAMA 290(7): 888-9 and Lane and Phillips, 2003, Brit. Med J. 327(7407): 115-6.

Rhabdomyolysis is a disorder caused by damage to muscle cells resulting in injury to the kidney. There are many conditions that can cause rhabdomyolysis including, for example, trauma and illicit drug use (e.g. cocaine, ecstasy, amphetamines or heroin) and use of medications such as statins. When muscle cells are damaged, myoglobin is released into the bloodstream and can occlude the kidneys resulting in acute renal failure or acute tubular necrosis. In addition to the renal complications of rhabdomyolysis, the necrotic skeletal muscle tissue can cause massive shifts of fluid from the blood into the muscle, resulting in low relative body fluids and increasing risk for shock. See, Wolfe, 2004, Lancet 363(9427): 2189-90; Davis and Bourke, 2004, Clin. Infect. Dis. 38(10): e109-11; Mansi and Huang, 2004, Am. J. Med. Sci. 327(6): 356-7; Guis et al., 2003, Best Pract. Res. Clin. Rheumatol. 17(6): 877-907; Nowicke, 2004, Crit. Care Nurse 23(2): 16; Criddle, 2003, Crit. Care Nurse 23(6): 14-22 and Wang et al., 2004, Ren. Fail. 26(1):93-7.

Recently, it was shown that the adverse effects of statins may be related to diminished biosynthesis of Coenzyme Q₁₀ and structurally related compounds. Coenzyme Q₁₀, a quinone compound that carries out oxidation and reduction processes within cells. It is thought that Coenzyme Q₁₀ deficiency is due to the statins' inhibitory effects on cholesterol synthesis, since both Coenzyme Q and cholesterol are produced in vivo via a common biosynthetic pathway. Rundek et al., 2004, Arch. Neurol. 61(6): 889-92.

Heart muscle strength is intimately tied to CoQ₁₀ levels, as CoQ₁₀ is tied to production of cellular ATP and thus, energy production. Most of the CoQ₁₀ in the human body is localized in the heart.

The compositions and methods of the present invention provide improved treatment, reduction and/or amelioration of hypercholesterolemia and cardiovascular disease. In certain embodiments, the invention provides a method of treating, reducing or ameliorating cardiovascular disease by administering a composition that includes a lipid-lowering agent and Coenzyme Q₁₀. In certain embodiments, the lipid-lowering agent can be an azetidinone (e.g., ezetimibe). In other exemplary embodiments, the lipid-lowering agent can be cholesteryl ester transfer protein (e.g., CETP).

### DETAILED DESCRIPTION OF EMBODIMENTS

The compositions and methods of the present invention provide for improved treatment, reduction or amelioration of cardiovascular disease. In certain embodiments, the invention provides a method the treating, reducing or ameliorating cardiovascular disease by administering a composition that includes a lipid-lowering agent and Coenzyme Q₁₀.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are petides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to the subject. Adminsitration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608 (1985); and Rossolini et al., 1994, Mol. Cell. Probes 8:91-98). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every polynucleotide sequence described herein, which encodes a protein also describes every possible silent variation, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid, which encodes a protein is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art.

One of skill will appreciate that many conservative variations of the fusion proteins and nucleic acid which encode the fusion proteins yield essentially identical products. For example, due to the degeneracy of the genetic code, “silent substitutions” (i.e., substitutions of a nucleic acid sequence which do not result in an alteration in an encoded protein) are an implied feature of every nucleic acid sequence which encodes an amino acid. Similarly, “conservative amino acid substitutions,” in one or a few amino acids in an amino acid sequence are substituted with different amino acids with highly similar properties (see, the definitions section, supra), are also readily identified as being highly similar to a particular amino acid sequence, or to a particular nucleic acid sequence which encodes an amino acid. Such conservatively substituted variations of any particular sequence are a feature of the present invention. See also, Creighton (1984) Proteins, W. H. Freeman and Company. In addition, individual substitutions, deletions or additions which alter, add or delete a single amino acid or a small percentage of amino acids in an encoded sequence are also “conservatively modified variations”.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1999 Supplement) (Ausubel). Suitable host cells for expression of recombinant polypeptides are known to those of skill in the art, and include, for example, eukaryotic cells including insect, mammalian and fungal cells (e.g., Aspergillus niger)

Examples of protocols sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Ihuis et al. eds) Academic Press Inc. San Diego, Calif. (1990) (Innis); Arnheim & Levinson (October 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e. C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, includes “alkylene” and those derivatives of alkyl defined in more detail below, such as “heteroalkyl.” Alkyl groups, which are limited to hydrocarbon groups, are termed “homoalkyl.”

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH=CH—O—CH₃, —Si(CH₃)₃, —CH₂—CH=N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. A “recombinant protein” is one which has been produced by a recombinant cell.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

A “heterologous sequence” or a “heterologous nucleic acid”, as used herein, is one that originates from a source foreign to the particular host cell, or, if from the same source, is modified from its original form. Thus, a heterologous glycoprotein gene in a eukaryotic host cell includes a glycoprotein-encoding gene that is endogenous to the particular host cell that has been modified. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

A “fusion protein” refers to a protein comprising amino acid sequences that are in addition to, in place of, less than, and/or different from the amino acid sequences encoding the original or native full-length protein or subsequences thereof.

Compositions

Coenzyme Q₁₀ refers to a series of quinones which are widely distributed in animals, plants and microorganisms. These quinones function in the biological electron transport systems responsible for energy conversion within living cells. Turunen et al., 2004, Biochim. Biophys Acta 1660(1-2): 171-99. Structurally, the Coenzyme Q group closely resembles tocopherols, vitamin E, members of the vitamin K family and tocopherylquinones (tocopherols with a quinone ring attached to the hydrocarbon tail.

Coenzyme Q, also termed ubiquinone, is implicated in a number of physiological functions and pathways including lipid-related gene induction, sepsis, hypertension and neurodegenerative diseases, including Parkinson's disease. See, for example, Gorelick et al., 2004, Am. J. Obstet. Gynecol. 190(5): 1432-4; Shults et al., 2004, Exp. Neurol. 188(2):491-4; L'Her and Sebert 2004, J. Lab. Clin. Med. 143(6): 352-7; Winkler-Stuck et al., 2004, J. Neurol. Sci. 220(1-2): 41-8; Wilburn et al., 2004, J. Clin. Hypertens. 6(5): 242-8; Beal 2004, Methods Enzymol. 382:473-87 and Sharma et al., 2004, Methods Enzymol. 382: 488-509.

The quinones of the Coenzyme Q₁₀ series, which are found in various biological species, differ only slightly in chemical structure and form a group of related, 2-3-dimethoxy-5-methyl-benzoquinones with a polyisoprenoid side chain in the 6-position which varies in length from 30 to 50 carbon atoms. Thus each isoprenoid unit in the chain contains five carbon atoms, the number of isoprenoid units in the side chain varies from 6 to 10. The different numbers of isoprenoid groups is designated by a subscript following the Q as in Q₁₀ (i.e., Coenzyme Q₁₀). The length of the side chain influences the properties of the Coenzyme Q. The members of the group known to occur naturally are Q₆ through Q₁₀. Coenzyme Q functions as an agent for carrying out oxidation and reduction within cells. Its primary site of function is in the terminal electron transport system where it acts as an electron or hydrogen carrier between the flavoproteins (which catalyze the oxidation of succinate and reduced pyridine nucleotides) and the cytochromes. This process, is carried out in the mitochondria of cells of higher organisms. In addition, coenzyme Q₁₀ has antioxidant and membrane stabilizing properties that serve to prevent cellular damage resulting from normal metabolic processes. Coenzyme Q plays an important role as an antioxidant to neutralize potentially damaging free radicals created in part by the energy-generating process.

As an energy carrier, coenzyme Q₁₀ is continually cycling through its oxidized and reduced states. In coenzyme Q₁₀'s reduced form (ubiquinol), the coenzyme Q₁₀ molecule holds electrons loosely and will quite easily transfer one or two electrons to neutralize free radicals. In its electron rich reduced form, coenzyme Q₁₀ is as potent an antioxidant as vitamin E. Coenzyme Q₁₀'s main role as an antioxidant is in the mitochondria where it first participates in the process by which free radicals are generated and then helps to quench the extra free radicals that threaten cellular components such as DNA, RNA, and cell membranes. One of coenzyme Q₁₀'s key antioxidant actions is within the cell membrane, where it counters the oxidative attack of polyunsaturated lipids (lipid peroxidation), which causes damage in a self-propagating, destructive chain reaction that ultimately results in membrane degeneration leading to cell death.

The structure of Coenzyme Q is provided below in both the oxidized (I) 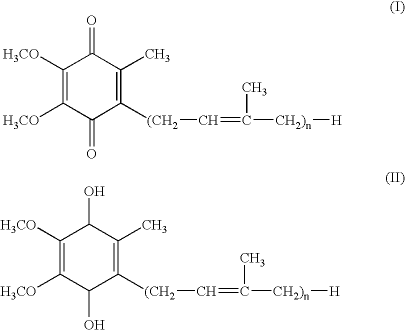
and reduced (II) forms. As will be apparent, a composition of the invention include a mixture of oxidized and reduced Coenzyme Q. As described above, in preferred embodiments, the number of isoprenoid units or, n, is 6-10 (i.e., Coenzyme Q₁₀)

In certain embodiments of the invention, the composition includes a lipid lowering agent combined with at least one ubiquinone. In an exemplary embodiment, the ubiquinone is Coenzyme Q. In a further exemplary embodiment, the ubiquinone is Coenzyme Q₁₀.

In another exemplary embodiment, the Coenzyme Q is prepared by the method described in commonly owned U.S. Pat. No. 6,545,184 or U.S. Provisional Patent Application No. 60/527,513.

Numerous lipid lowering agents are available and known to those skilled in the art and within the scope of the present invention. In certain embodiments, the lipid lowering agent can be, for example, a bile acid resin, fibrate, nicotinic acid, azetidinone, analogs, derivatives or combinations of these species. See, Drug Facts and Comparisons, updated monthly, Wolter Kluwer Company, St. Louis, Mo. and Physicians' Desk Reference. 54th Ed., Medical Economics Company, Montvale, N.J., 2000.

In exemplary embodiments, the bile acid resin is cholestyramine (QUESTRAN®, Par, PREVALITE®, Upsher Smith), colestipol (CHOLESTID®, Pharmacia) or colesevelam (WELCHOL®, Sankyo Pharma). In other exemplary embodiments, the fibrate, or fibric acid derivative is gemfibrozil (LOPID®, Pfizer) or fenofibrate (TRICOR®, Abbott). In another exemplary embodiment nicotinic acid (e.g., niacin) can be in a rapid release or extended or sustained release forms, for example NIASPAN® (Kos).

In other exemplary embodiments, the lipid lowering agent is a statin. As is known to those skilled in the art, statins are also termed HMG-CoA reductase due to their pharmacologic activity; they act by competitively inhibiting 3-hydroxyl-3-methyl-glutaryl-coenzyme A reductase. Exemplary statins of use in the invention include atorvastatin (LIPITOR®, Pfizer), fluvastatin (LESCOL® Novartis), lovastatin (MEVACOR®, Merck), pravastatin (PRAVACOL®, BristolMeyersSquibb), rosuvastatin (CRESTOR®, AstraZeneca) or simvastatin (ZOCOR®, Merck).

In certain embodiments the lipid lowering agent is an azetidinone, (e.g., ezetimibe, ZETIA®, Merck/Schering Plough, U.S. Pat. No. 5,767,115, incorporated herein by reference in its entirety). In an exemplary embodiment, the lipid lowering agent can be an analog or derivative of the lipid lowering agents described herein. In another exemplary embodiment, the lipid lowering agent is a combination of the lipid lowering agents described herein (e.g., ADVICOR®, Kos, a combination of extended release niacin with lovastatin) or combinations of an analog or derivative of the lipid lowering agents described herein.

In an exemplary embodiment, the invention provides a composition that includes a ubiquinone in combination with more than one lipid lowering agent. In one exemplary embodiment, the invention provides for a composition including a ubiquinone (e.g., Coenzyme Q₁₀), a non-statin lipid lowering agent (e.g., ezetimibe) and a statin (e.g., simvastatin). In another exemplary embodiment, the invention provides for a composition including a ubiquinone (e.g., Coenzyme Q₁₀), a non-statin lipid lowering agent (e.g., a reverse cholesterol transport protein inhibitors (torcetrapib) and a statin (e.g., simvastatin). In yet another exemplary embodiment, the invention provides for a composition including a ubiquinone (e.g., Coenzyme Q₁₀), two non-statin lipid lowering agents (e.g., ezetimibe and CETP) and a statin (e.g., simvastatin).

In certain embodiments, the azetidinone can be of the formula provided below: 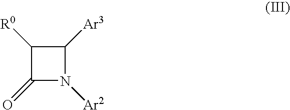
in which R° is substituted or unsubstituted alkyl or substituted or unsubstituted heteroalkyl, or a pharmaceutically acceptable salt thereof. In Formula III, Ar² and Ar³ independently represent aryl groups.

In a still further exemplary embodiment, the lipid lowering agent has the formula: 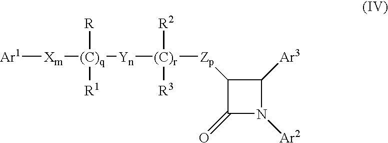
In which Ar¹, Ar² and Ar³ independently represent aryl groups. The symbols X, Y and Z independently represent a bond, substituted or unsubstituted alkyl or substituted or unsubstituted heteroalkyl.

The symbols R and R² are independently selected from the group consisting of —OR⁶, —OC(O)R⁶, —OC(O)OR⁹ and —OC(O)NR⁶ R⁷. The symbols R¹ and R³ independently represent hydrogen, substituted or unsubstituted alkyl or substituted or unsubstituted aryl.

The index q is 0 or 1. The index r is 0 or 1. The indexes m, n and p are independently 0, 1, 2, 3 or 4. In an exemplary embodiment, at least one of q and r is 1, and the sum of m, n, p, q and r is 2, 3, 4, 5 or 6. In another exemplary embodiment, when p is 0 and r is 1, the sum of m, q and n is 1, 2, 3,4 or 5.

Exemplary substituents for the aryl group include substituted or unsubstituted alkyl, —OR⁶, —OC(O)R⁶, —OC(O)OR⁹, —O(CH₂)₅—, OR⁶, —OC(O)NR⁶R⁷, —NR⁶R⁷, —COR⁶, —NR⁶C(O)OR⁹, —NR⁶C(O)NR⁷R⁸, —NR⁶SO₂R⁹, —C(O)OR⁶, —CONR⁶R⁷, —COR⁶, —SO₂NR⁶R⁷, —S(O)₀₋₂R⁹, —O(CH₂)₁₋₁₀—C(O)OR⁶, —O(CH₂)₁₋₁₀C(O)NR⁶R⁷, —CH═CH—C(O)OR⁶, —CF₃, —CN, —NO₂ and halogen.

The symbol R⁶, R⁷ and R⁸ independently represent hydrogen, substituted or unsubstituted alkyl or substituted or unsubstituted aryl.

The symbol R⁹ represents substituted or unsubstituted alkyl or substituted or unsubstituted aryl.

In a preferred embodiment, the azetidinone is ezetimibe, or an analogue, or pharmaceuticall acceptable salt thereof. The structure of ezetimibe is provided in formula V below:

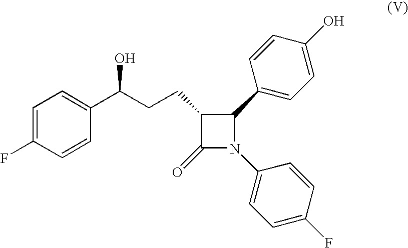

Ezetimibe has the chemical name 1-(4-fluorophenyl)-3-(R)-[3(S)-(4-fluorophenyl)-3-hydroxypropyl-4(S)-(4-hydroxyphenyl)]-2-azetidinone. Catapano, 2001, Eur. Heart J. Suppl. 3: (suppl E): E6-E10. The compound localizes at the brush border of the small intestine to prevent and decrease the delivery of intestinal cholesterol in the liver. The reduction of hepatic cholesterol stores leads to an increase in clearance of cholesterol from the blood. van Heek et al., 2000 Br. J. Pharmacol. 129: 1748-1754. Upon ingestion, the compound is rapidly metabolized to an active glucuronide metabolite. The enterohepatic recirculation of glucuronide metabolite extends the duration of action of the compound. Bays et al., 2001, Clin. Ther. 23: 1209-1230; Kirsten et al., 1998, Clin. Pharmacokinet. 34: 457-482; Patrick et al. 2002, Drug Metab Dispos. 30: 430-437 and Ezzett et al. 2001, Clin. Ther. 23: 871-885. Other agents that function to reduce lipid levels in the blood stream according to this mechanism are of use in the present invention.

In yet another exemplary embodiment, the invention provides compositions of species such as Caduet, Norvasc, and Avasimbe in conjunction with Coenzyme Q.

In certain embodiments, the compositions and methods of the present invention provide for increased or improved cholesterol or lipid removal from body. Reverse cholesterol transport (RCT) is the pathway by which peripheral cell cholesterol can be returned to the liver for recycling to extrahepatic tissues, or excretion into the intestine in bile, either in modified or in oxidized form as bile acids. The RCT pathway is a means of eliminating cholesterol from most extrahepatic tissues, and is crucial to maintenance of the structure and function of most cells in the body.

The RCT consists mainly of three steps: (a) cholesterol efflux, the initial removal of cholesterol from various pools of peripheral cells; (b) cholesterol esterification by the action of lecithin:cholesterol acyltransferase (LCAT), preventing a re-entry of effluxed cholesterol into cells; and (c) uptake/delivery of HDL cholesteryl ester to liver cells. The RCT pathway is mediated by high density lipoproteins (HDLs).

Several enzymes are involved in the RCT pathway including LCAT, cholesteryl ester transfer protein (CETP) and phospholipid transfer protein (PLTP). LCAT is produced mainly in the liver and circulates in plasma associated with the HDL fraction. LCAT converts cell derived cholesterol to cholesteryl esters which are sequestered in HDL destined for removal. CETP and PLTP contribute to further remodeling the circulating HDL population. CETP can move cholesteryl esters made by LCAT to other lipoproteins, particularly ApoB-containing lipoproteins, such as very low density lipoproteins (VLDL) and low density lipoproteins (LDL). PLTP supplies lecithin to HDL. HDL triglycerides can be catabolized by the extracellular hepatic triglyceride lipase, and lipoprotein cholesterol is removed by the liver via several mechanisms.

CETP facilitates the exchange of lipids between HDL and ApoB containing lipoproteins thereby playing a role in “remodeling” the HDL population. The protein's exact role as a promoter or protector from atherosclerosis has yet to be elucidated and data is conflicting. Ritsch and Patsch 2003, Curr. Opin. Lipidol. 14(2): 173-9, Hogue et al., 2004, J Lipid Res., 2004;45(6):1077-1083. However, recently torcetrapib, an inhibitor of CETP, was found to raise HDL cholesterol. Subjects in this study had an increased plasma concentration of HDL cholesterol by 61%. Brousseau et al., 2004, N. Engl. J. Med. 350:1505-1515.

Human CETP consists of 476 amino acids. The CETP gene is in chromosome band 16q21. A number of mutations and polymorphs are known in the CETP gene. See, LeGoff et al., 2002, Atherosclerosis 161(2):269-79; Thompson et al., 2004, Clin. Genetics 0(0), doi: 10.1111/j.1399-0004.2004.00289; van Venrooij et al., 2003, Diabetes Care, April. The cDNA sequence for tree shrew (Tupaia glis) CETP, a model for insusceptibility to atherosclerosis, has been determined. Zeng et al., 2003, Chin. Med. 116(6): 928-931, GenBank accession number AF334033. The sequence has 1636 base pairs, 1458 base pairs in the coding region. The first 24 base pairs encode a partial signal peptide. Tree shrew CETP is extremely hydrophobic, containing many hydrophobic amino acid residues, especially at the C-terminus. This hydrophobic nature, especially at the C-terminus, is consistent with its function in the transfer of neutral lipids.

In another exemplary embodiment, a CoQ is combined with a mutant of wild type CETP. A preferred mutant is one that includes one or more conservative substitutions in the CETP sequence. Other useful mutants include truncations and deletions. Methods of making mutant peptides through recombinant technology, starting with a desired nucleic acid, are well known in the art.

In yet another exemplary embodiment, the lipid lowering agent can be an agent that increases HDL or “good cholesterol” as described, infra. In one embodiment, the lipid lowering agent is one which inhibits cholesteryl ester transfer protein (CETP). In an exemplary embodiment, the lipid lowering agent can be torcetrapib., the structure of which is provided in formula VI below:

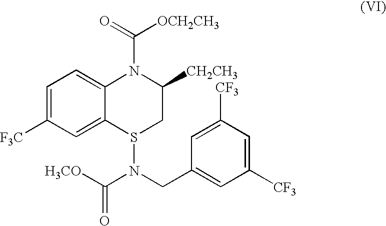

The compositions described herein are preferably pharmaceutical compositions formulated according to known methods. Formulations are described in detail in a number of sources, which are well known and readily available to those skilled in the art. For example, Remington's Pharmaceutical Science by E. W. Martin describes formulation, which can be used in connection with the subject invention. In general, the compositions of the subject invention are formulated such that an effective amount of the lipid lowering agent and coenzyme Q are provided in the composition.

In accordance with the present invention, pharmaceutical compositions are provided which comprise, an active ingredients as described, supra, and an effective amount of one or more pharmaceutically acceptable excipients, vehicles, carriers or diluents. Examples of such carriers include ethanol, dimethyl sulfoxide, glycerol, silica, alumina, starch, and equivalent carriers and diluents. Further, acceptable carriers can be either solid or liquid. Solid form preparations include powders, tablets, pills, capsules, cachets, suppositories and dispersible granules. A solid carrier can be one or more substances, which may act as diluents, flavoring agents, solubilizers, lubricants, suspending agents, binders, preservatives, tablet disintegrating agents or encapsulating materials.

Injectable preparations include sterile suspensions, solutions or emulsions of the active ingredient in aqueous or oily vehicles. The compositions can also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the compositions can be lyophilized. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection. Thus, for example, the active ingredient can be formulated with suitable polymeric or hydrophobic materials (e.g., as an emulsion in an acceptable oil) or ion exchange resins, or as sparingly soluble derivatives; e.g., as a sparingly soluble salt form of the lipid proteins.

Alternatively, transdermal delivery systems manufactured as an adhesive disc or patch which slowly releases the active ingredient for percutaneous absorption can be used. To this end, permeation enhancers can be used to facilitate transdermal penetration of the active ingredient. A particular benefit can be achieved by incorporating the active agents of the invention into a nitroglycerin patch for use in patients with ischemic heart disease and hypercholesterolemia.

For oral administration, the pharmaceutical compositions can take any appropriate form, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal administration, the compositions can take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. Also, the unit dosage can be a liquid based preparation or formulated to be incorporated into solid food products, chewing gum, or lozenges.

Methods of Treatment

The present invention provides methods for the treatment, reduction or amelioration of cardiovascular disease. Cardiovascular disease refers to diseases which affect the proper functioning of the heart and blood vessels. Primary among these are myocardial infarction (heart attack), cerebrovascular disease (stroke), transient ischemic attacks (TIAs) and peripheral vascular disease (PVDs). See, for example World Health Organization at http://www.who.int/cardiovascular_diseases. Other forms of cardiovascular disease include coronary artery disease, acute coronary syndrome and angina pectoris, (stable or unstable). See, generally, Merck Manual, 17th ed. Beers and Berkow, eds. Merck Research Laboratories, Whitehouse Station, N.J., 1999.

Atherosclerosis is a significant contributor to cardiovascular disease. Lowering cholesterol levels can arrest or reverse atherosclerosis in all vascular beds and can significantly decrease the morbidity and mortality associated with atherosclerosis. Each 10% reduction in cholesterol levels is associated with about a 20 to 30% reduction in the incidence of coronary heart disease. Hyperlipidemia, particularly elevated serum cholesterol and low-density lipoprotein (LDL) levels, is a risk factor in the development of atherosclerotic cardiovascular disease.

Although most subjects require drug therapy for control of primary hyperlipidemia, diet restriction, weight reduction and reduction or elimination of alcohol consumption are also recommended.

Elevated blood cholesterol levels are a major cause of coronary artery disease. Lowering these levels, specifically, LDL will reduce the risk of heart attacks caused by coronary heart disease (CHD).

The National Cholesterol Education Program Expert Panel on Detection, Evaluation and Treatment of High Blood Cholesterol in Adults has set the following guidelines for therapy in adults ≧20 years of age based on LDL and HDL levels.

Classification of Total and HDL-Cholesterol Levels
(Adults ≧20 years of Age)

| Level (mg/cl) | (mmol/L) | Classification |
|---|---|---|
| <200 | (5.2) | desirable |
| 200-239 | (5.2-6.2) | borderline-high |
| ≧240 | (6.2) | high |
| HDL < 35 | (0.9) | low |

Classification of LDL-Cholesterol Levels

| Level (mg/dl) | (mmol/L) | Classification |
|---|---|---|
| <130 | (3.4) | desirable |
| 130-159 | (3.4-4.1) | borderline-high |
| ≧160 | (4.1) | high |

Cardiovascular disease can be asymptomatic. Symptoms, when present, include pallor, diaphoresis, pain (often crushing or squeezing the chest and radiating to the neck, jaw, shoulder or arm), nausea, vomiting, palpitations, tachycardia, weakness, fatigue and light-headedness. Objective indicators include elevations in serum enzyme levels, (e.g., troponin (TnT and Tnl), creatine phosphokinase (CK or CPK and isoenzymes MM, MB and BB), lactate dehydrogenase (LDH) and myoglobin). See, for example, Stoffel et al., 2000, Nephrol. Dial. Transplant. 15: 1259-60; Thijssen et al., 2004, Circulation 110(7): 770-5; Svensson et al., 2004, J. Intern. Med. 255(4): 469-77; Roe et al., 2004, Eur. Heart J. 25(4): 313-21 and Miller et al., 2004, Chest 125(1): 275-80.

Other diagnostic aids include electrocardiography (EKG or ECG), radiography, radionuclide imaging (e.g., Tc^{99m} perfusion imaging), stress testing, echocardiography, positron emission tomography (PET), magnetic resonance imaging (MRI) and catheterization. Merck Manual, 17th ed. Beers and Berkow, eds. Merck Research Laboratories, Whitehouse Station, N.J., 1999; Mihelic et al., 2002, J. Insur. Med. 34(1): 12-25; ten Wolde et al., 2004, Arch. Intern. Med. 164(15): 1685-9; Nasir, et al., 2004, Arch. Intern. Med. 164(15): 1610-20; Perschbacher et al., 2004, Mayo Clin. Proc. 79(8): 983-9 and Polanczyk and Lee, 2004, Am. J. Med. 117(3): 200-1.

The methods of the invention provide for administration of a composition, described supra, with a dose, dosing schedule and dosing duration sufficient to treat, prevent or ameliorate cardiovascular disease or symptoms thereof. In an exemplary embodiment, the dose of Coenzyme Q can be about 0.1 mg to about 4,000 mg. In another exemplary embodiment, the dose of Coenzyme Q can be abut 20 mg, about 50 mg, about 100 mg, about 200 mg, about 500 mg, about 1,000 mg, about 1,200 mg, about 1,500 mg or about 2,000 mg.

In another exemplary embodiment, the dose of lipid lowering agent can be one which imparts a therapeutic or prophylactic effect on the subject. When the lipid lowering agent is an azetidinone, e.g., ezetimibe, exemplary doses are about 0.5 mg, about 1 mg., about 2 mg, about 3 mg, about 4 mg, about 5 mg, about 6 mg, about 7 mg, about 8 mg, about 9 mg, about 10 mg, about 12 mg, about 15 mg or about 20 mg per day for a 70 kg patient.

When the lipid lowering agent is a statin, exemplary doses can be about 0.5 mg, about 1 mg., about 2 mg, about 5 mg, about 8 mg, about 10 mg, about 12 mg, about 15 mg, about 20 mg, about 25 mg, about 30 mg, about 35 mg, about 40 mg, about 45 mg, about 50 mg, about 55 mg or about 60 mg per day for a 70 kg patient.

The methods of the invention provide for administration of a composition on a dosing schedule that provides a therapeutic or prophylactic amount of the composition. In an exemplary embodiment, the composition can be administered about four times daily, about three times daily, about twice daily, about daily, about every other day, about three times weekly, about twice weekly, about weekly or about every two weeks.

The methods of the invention provide that the composition can be administered for a duration sufficient to provide a therapeutic or prophylactic effect. In an exemplary embodiment, the composition can be administered, for example, daily for one year. In another embodiment, the composition can be administered for about six months, about a year, about two years, about five years, about 10 years or indefinitely. In a particularly preferred embodiment, the composition can be administered daily for five years.

It will be apparent to those of skill in the art that the dose, dosing schedule and duration can be adjusted for the needs of the particular subject taking into consideration the subject's age, weight, severity of disease and other co-morbid conditions.

1. A composition comprising a ubiquinone and an azetidinone.

2. The composition of claim 1 wherein the ubiquinone is Coenzyme Q.

3. The composition of claim 2 wherein the Coenzyme Q is Coenzyme Q₁₀.

4. The composition of claim 1 wherein the azetidinone is 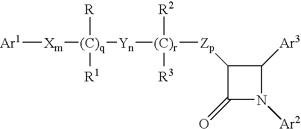
or a pharmaceutically acceptable salt thereof, wherein:
Ar¹, Ar² and Ar³ are independently selected from the group consisting of aryl and R⁴-substituted aryl;
X, Y and Z are independently selected from the group consisting of a bond, substituted or unsubstituted alkyl or substituted or unsubstituted heteroalkyl;
R and R² are independently selected from the group consisting of —OR⁶, —O(CO)R⁶, —OC(O)OR⁹ and —OC(O)NR⁶R⁷;
R¹ and R³ are independently selected from the group consisting of hydrogen, substituted or unsubstituted alkyl and substituted or unsubstituted aryl;
q is 0 or 1;
r is 0 or 1;
m, n and p are independently 0, 1, 2, 3 or 4; and
provided that at least one of q and r is 1, and the sum of m, n, p, q and r is 2, 3, 4, 5 or 6;
and provided that when p is 0 and r is 1, the sum of m, q and n is 1, 2, 3, 4 or 5.

5. The composition according to claim 4 wherein a member selected from Ar¹, Ar², Ar³ and combinations thereof is substituted with one or more group which is a member selected from:
substituted or unsubstituted alkyl, —OR⁶, —OC(O)R⁶, —OC(O)OR⁹, —O(CH₂)₁₋₅R⁶, —OC(O)NR⁶R⁷, —NR⁶R⁷, —NR⁶C(O)R⁷, —NR⁶C(O)OR⁹, —NR⁶C(O)N⁷R⁸, —NR⁶—SO₂R⁹, —C(O)OR⁶, —C(O)NR⁶R⁷, —COR⁶, —SO₂NR⁶R⁷, —S(O)₀₋₂R⁹, —O(CH₂)₁₋₁₀—O(CH₂)₁₋₁₀CONR⁶R⁷, —CH═CH—C(O)OR⁶, —CF₃, —CN, —NO₂ and halogen in which
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

6. The composition according to claim 5 wherein Ar¹, Ar² and Ar³ are independently selected substituted or unsubstituted phenyl moieties.

7. The composition of claim 5 wherein Ar¹ is R⁴-substituted phenyl wherein R⁴ is halogen; Ar² is R⁴-substituted phenyl wherein R⁴ is halogen or —OR⁶, wherein R⁶ is lower alkyl or hydrogen; and Ar³R⁵-substituted phenyl, wherein R⁵ is —OR⁶, wherein R⁶ is lower alkyl or hydrogen.

8. The composition of claim 5 wherein m, n and r are each zero, q is 1 and p is 2.

9. The composition of claim 5 wherein p, q and n are each zero, r is 1 and m is 2 or 3.

10. The composition of claim 5 wherein the azetidinone is ezetimibe.

11. The composition of claim 2 wherein the Coenzyme Q is in oxidized or reduced form.

12. The composition of claim 8 wherein the Coenzyme Q is CoenzymeQ₁₀.

13. The composition of claim 11 having 0.1 mg to 4,000 mg of Coenzyme Q₁₀ in each dosage unit.

14. The composition of claim 1 which is an injectable dosage form.

15. The composition of claim 14 which is suitable for intravenous, intramuscular, subcutaneous or pericardial administration.

16. The composition of claim 1 which is a topical dosage form.

17. The composition of claim 16 which is a topical solution, topical suspension, ointment, cream, lotion or transdermal patch.

18. The composition of claim 1 which is a sustained release dosage form.

19. A composition comprising Coenzyme Q and cholesteryl ester transfer protein (CETP) inhibitor.

20. The composition of claim 19 wherein the Coenzyme Q is in an oxidized or reduced form.

21. The composition of claim 20 wherein Coenzyme Q is Coenzyme Q₁₀.

22. The composition of claim 19 which is an injectable dosage form.

23. The composition of claim 22 which is suitable for intravenous, intramuscular, subcutaneous or pericardial administration.

24. The composition of claim 19 which is a topical dosage form.

25. The pharmaceutical composition of claim 24 which is a topical solution, topical suspension, ointment, cream, lotion or transdermal patch.

26. The composition of claim 19 which is a sustained release dosage form.

27. The composition of claim 19 further comprising an azetidinone.

28. The composition of claim 27 wherein the azetidinone is 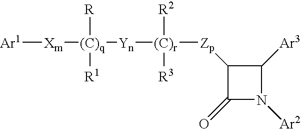
or a pharmaceutically acceptable salt thereof, wherein:
Ar¹, Ar² and Ar³ are independently selected from the group consisting of aryl and R⁴-substituted aryl;
X, Y and Z are independently selected from the group consisting of a bond, substituted or unsubstituted alkyl or substituted or unsubstituted heteroalkyl;
R and R² are independently selected from the group consisting of —OR⁶, —O(CO)R⁶, —OC(O)OR⁹ and —OC(O)NR⁶R⁷;
R¹ and R³ are independently selected from the group consisting of hydrogen, substituted or unsubstituted alkyl and substituted or unsubstituted aryl;
q is 0 or 1;
r is 0 or 1;
m, n and p are independently 0, 1, 2, 3 or 4; and
provided that at least one of q and r is 1, and the sum of m, n, p, q and r is 2, 3, 4, 5 or 6;
and provided that when p is 0 and r is 1, the sum of m, q and n is 1, 2, 3, 4 or 5.

29. The composition according to claim 28 wherein a member selected from Ar¹, Ar², Ar³ and combinations thereof is substituted with one or more group which is a member selected from:
substituted or unsubstituted alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —C(O)OR⁶, —C(O)NR⁶R⁷, —COR⁶, —SO₂NR⁶R⁷, —S(O)₀₋₂R⁹, —O(CH₂)₁₋₁₀—O(CH₂)₁₋₁₀CONR⁶R⁷, —CH═CH—C(O)OR⁶, —CF₃, —CN, —NO₂ and halogen in which
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

30. The composition according to claim 29 wherein Ar¹ is substituted or unsubstituted phenyl, Ar² is substituted or unsubstituted phenyl and Ar³ is substituted or unsubstituted phenyl.

31. The composition of claim 29 wherein Ar¹ is R⁴-substituted phenyl wherein R⁴ is halogen; Ar² is R⁴-substituted phenyl wherein R⁴ is halogen or —OR⁶, wherein R⁶ is lower alkyl or hydrogen; and Ar³ R⁵-substituted phenyl, wherein R⁵ is —OR⁶, wherein R⁶ is lower alkyl or hydrogen.

32. The composition of claim 29 wherein m, n and r are each zero, q is 1 and p is 2.

33. The composition of claim 29 wherein p, q and n are each zero, r is 1 and m is 2 or 3.

34. The composition of claim 29 wherein the azetidinone is ezetimibe.

35. A method of treating a subject afflicted with atherosclerosis or coronary heart disease comprising administering to said subject an effective amount of a composition comprising an azetidinone and coenzyme Q.

36. The method of claim 35 wherein the Coenzyme Q is in the oxidized or reduced form.

37. The method of claim 36 wherein the Coenzyme Q is Coenzyme Q₁₀.

38. The method of claim 35 wherein the Coenzyme Q is administered at a dose of 0.1 mg to 4,000 mg.

39. The method of claim 35 wherein the composition is administered once daily to four times daily.

40. The method of claim 35 wherein the composition is administered orally.

41. The method of claim 35 wherein the subject has plaque deposits in the blood vessels.

42. The method of claim 35 wherein the subject has an elevated serum LDL, triglyceride or cholesterol levels.

43. The method of claim 35 wherein the subject has a decreased serum HDL level.

44. The method of claim 35 wherein myopathy or rhabdomyolysis is decreased or avoided in the subject.

45. A method of treating a subject afflicted with atherosclerosis or coronary heart disease comprising administering to said subject an effective amount of a composition comprising Coenzyme Q and cholesteryl ester transfer protein inhibitor.

46. The method of claim 45 wherein the Coenzyme Q is administered at a dose of 0.1 mg to 4,000 mg.

47. The method of claim 45 wherein the composition is administered once daily to four times daily.

48. The method of claim 45 wherein the subject has plaque deposits in the blood vessels.

49. The method of claim 45 wherein the subject has an elevated serum LDL, triglyceride or cholesterol level.

50. The method of claim 45 wherein the subject has a decreased serum HDL serum level.
